(19) United States
(12) Patent Application Publication
(10) Pub. No.: US 2016/0193369 A1
(43) Pub. Date: Jul. 7, 2016

(54) MAGNETIC NANOPARTICLE AND METHOD FOR IMAGING T CELLS

(71) Applicants: University of Washington, Seattle, WA (US); Fred Hutchinson Cancer Research Center, Seattle, WA (US)

(72) Inventors: Miqin Zhang, Bothell, WA (US); Jonathan Whitney Gunn, Seattle, WA (US); Cassian Yee, Seattle, WA (US)

(73) Assignees: University of Washington, Seattle, WA (US); Fred Hutchinson Cancer Research Center, Seattle, WA (US)

(21) Appl. No.: 15/066,717

(22) Filed: Mar. 10, 2016

Related U.S. Application Data
(62) Division of application No. 12/901,271, filed on Oct. 8, 2010, now abandoned.
Application No. 12/901,271 is a continuation of application No. PCT/US2009/040114, filed on Apr. 9, 2009.
(60) Provisional application No. 61/043,596, filed on Apr. 9, 2008.

(51) Int. Cl. A61K 49/14; G01N 33/569; G01N 33/543; A61K 49/18

(52) U.S. Cl. CPC A61K 49/14; A61K 49/1818; G01N 33/56972; G01N 33/54346; G01N 33/54326; G01N 2333/7051

(57) ABSTRACT

The present invention provides nanoparticles having a core comprising a magnetic material and having a surface, where the surface may be operatively linked to an antigenic peptide-major histocompatibility complex (MHC) monomer. The antigenic peptide-MHC monomer may then be recognized by a T cell receptor. These nanoparticles may further comprise a signal-generating label, such as a fluorophore. Methods employing nanoparticles of the present invention may involve magnetic resonance imaging and/or fluorescence detection, such that cell imaging and localization are performed.

### CROSS-REFERENCES TO RELATED APPLICATIONS

This application is a divisional of U.S. patent application Ser. No. 12/901,271, filed Oct. 8, 2010, which is a continuation of International Patent Application No. PCT/US2009/040114, filed Apr. 9, 2009, which claims the benefit of U.S. Provisional Application No. 61/043,596, filed Apr. 9, 2008, all of which are hereby incorporated by reference in their entirety.

### STATEMENT OF GOVERNMENT LICENSE RIGHTS

This invention was made with government support under R01 CA119408, R01 EB006043, and T32 GM065098, awarded by the National Institutes of Health. The government has certain rights in the invention.

### BACKGROUND

Cancer immunotherapy approaches, including vaccination, adoptive cell transfer (ACT), and combinational strategies, have been developed to assist the body's immune system to selectively recognize and kill malignant tumor cells. Currently, immunotherapies are evaluated by either function-based assays, such as enzyme-linked immunosorbent spot (ELISPOT) and limiting dilution studies, or structure-based assays such as peptide-MHC tetramer labeling. These assessment methods require invasive sample collection, have not yielded strong correlations with clinical responses to treatment, and provide limited in vivo T cell tracking information. Alternative visualization strategies have been developed, whereby T cells extracted from an animal and labeled ex vivo are injected back into the animal to be monitored. This approach has been applied to positron emission tomography (PET), single-photon emission computed tomography (SPECT), and multi-photon intravital microscopy.

More recently, magnetic nanoparticle labeling of cells for in vivo tracking by magnetic resonance imaging (MRI) has received considerable attention, as MRI offers superior capabilities for deep-tissue, whole-body imaging at higher resolution than alternative imaging modalities. The MRI method is an imaging method that involves exciting nuclear spins in tissues of a subject placed in a static magnetic field with a radio-frequency signal (RF pulses) having their Larmor frequency and reconstructing image data from magnetic resonance signals emitted as a result of the nuclear spins having been excited. MRI can obtain not only anatomical diagnostic information of a subject, but also biochemical information and diagnostic function information. For these reasons, MRI plays an increasingly important role in the field of imaging and diagnosis.

Magnetic nanoparticles have been coupled with immunotherapy regimens as ex vivo T cell labels for ACT, inducing non-specific cellular uptake through conjugation with the transmembrane HIV-Tat peptide, poly-L-lysine, or by using lipofection reagents. While capable of labeling cells, these nanoparticles cannot specifically bind to cytotoxic T lymphocytes (CTLs) (which are cells that destroy virally infected cells and tumor cells), and thus use of these nanoparticles in vitro requires either CTL isolation or prolonged CTL expansion before the labeling can be performed, and for in vivo tracking is limited to externally tagged cells, neglecting endogenously recruited, vaccine-elicited, or ad hoc labeling of adoptively transferred CTLs. Further developments in magnetic nanoparticle technology are needed to minimize or eliminate these drawbacks.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

The present invention provides a nanoparticle system capable of selectively labeling and imaging cells expressing T cell receptors that recognize cognate MHC-peptide complexes on the surface of antigen-presenting cells, such as tumor cells. Accordingly, in one aspect, the present invention contemplates a nanoparticle comprising: (a) a core comprising a magnetic material and having a surface; and (b) an antigenic peptide-major histocompatibility complex (MHC) monomer operatively linked to the surface, wherein the antigenic peptide-MHC monomer is recognized by a T cell receptor.

In certain embodiments, a nanoparticle may comprise (a) a core comprising a magnetic material and having a surface coated with a polymer; and (b) an antigenic peptide-major histocompatibility complex (MHC) monomer operatively linked to the polymer, wherein the antigenic peptide-MHC monomer is recognized by a T cell receptor.

Nanoparticles are also contemplated by the present invention that comprise: (a) a core comprising a magnetic material and having a surface covalently bound to a polymer; (b) a biotinylated antigenic peptide-major histocompatibility complex (MHC) monomer operatively linked to the polymer, wherein the antigenic peptide-MHC monomer is recognized by a T cell receptor; and (c) an avidin protein that is bound to the antigenic peptide-MHC monomer through a biotin/avidin interaction and is also covalently bound to the polymer.

Another aspect of the present invention contemplates a nanoparticle, comprising: (a) a core comprising a magnetic material and having a surface; (b) an antigenic peptide-major histocompatibility complex (MHC) monomer operatively linked to the surface, wherein the antigenic peptide-MHC monomer is recognized by a T cell receptor; and (c) a fluorophore.

In certain embodiments, a nanoparticle may comprise (a) a core comprising a magnetic material and having a surface covalently bound to a polymer; (b) an antigenic peptide-major histocompatibility complex (MHC) monomer operatively linked to the polymer, wherein the antigenic peptide-MHC monomer is recognized by a T cell receptor; (c) an avidin protein operatively linked to the antigenic peptide-MHC monomer and also covalently bound to the polymer; and (d) a fluorophore.

A composition comprising a nanoparticle as described herein and a pharmaceutically acceptable carrier, excipient or diluent, suitable for administration to a subject, is another embodiment of the present invention.

Methods employing nanoparticles of the present invention are also contemplated. For example, in certain embodiments, the present invention contemplates a method of detecting the presence of cells having a T cell receptor in a sample, comprising: (a) contacting the sample with a nanoparticle comprising: (i) a core comprising a magnetic material and having a surface; and (ii) an antigenic peptide-major histocompatibility complex (MHC) monomer operatively linked to the surface, wherein the antigenic peptide-MHC monomer is recognized by the T cell receptor; and (b) measuring the level of nanoparticle binding to cells in the sample using magnetic resonance imaging.

Methods of detecting the presence of cells having a T cell receptor in a subject are also contemplated, wherein such methods may comprise: (a) administering to the subject a nanoparticle comprising: (i) a core comprising a magnetic material and having a surface; and (ii) an antigenic peptide-major histocompatibility complex (MHC) monomer operatively linked to the surface, wherein the antigenic peptide-MHC monomer is recognized by the T cell receptor; and (b) measuring the level of nanoparticle binding to cells in the subject using magnetic resonance imaging.

In other embodiments, the present invention contemplates a method of detecting the presence of T cells having a T cell receptor in a subject, comprising: (a) removing T cells from a subject; (b) performing expansion of the T cells; (c) contacting the expanded T cells with a nanoparticle comprising: (i) a core comprising a magnetic material and having a surface; and (ii) an antigenic peptide-major histocompatibility complex (MHC) monomer operatively linked to the surface, wherein the antigenic peptide-MHC monomer is recognized by the T cell receptor; (d) introducing the expanded T cells that have been contacted with a nanoparticle as in step (c) back into the subject; and (e) measuring the level of nanoparticle binding to the T cells in the subject using magnetic resonance imaging.

The present invention further contemplates a method of detecting the presence of cells having a T cell receptor in a sample, comprising: (a) contacting the sample with a nanoparticle comprising: (i) a core comprising a magnetic material and having a surface; (ii) an antigenic peptide-major histocompatibility complex (MHC) monomer operatively linked to the surface, wherein the antigenic peptide-MHC monomer is recognized by the T cell receptor; and (iii) a fluorophore; (b) isolating those cells from the sample that bound to a nanoparticle; and (c) measuring the level of nanoparticle binding to cells in the sample using fluorescence detection.

Methods of determining the localization of a nanoparticle in a cell are also contemplated by the present invention. Such methods may comprise, for example: (a) contacting the cell with a nanoparticle, wherein the nanoparticle comprises: (i) a core comprising a magnetic material and having a surface; (ii) an antigenic peptide-major histocompatibility complex (MHC) monomer operatively linked to the surface, wherein the antigenic peptide-MHC monomer is recognized by a T cell receptor; and (iii) a fluorophore; and (b) detecting the location of the nanoparticle in the cell.

In yet further embodiments, the present invention contemplates a method of making a nanoparticle, comprising: (a) obtaining a core comprising a magnetic material and having a surface; (b) coating the surface with a polymer; (c) covalently coupling an avidin protein to the polymer to form a core-polymer-avidin protein complex; (d) biotinylating an antigenic peptide-major histocompatibility complex (MHC) monomer; and (e) coupling the core-polymer-avidin protein complex to the biotinylated antigenic peptide-MHC monomer.

### DESCRIPTION OF THE DRAWINGS

The patent or application file contains at least one drawing executed in color. Copies of this patent or patent application publication with color drawing(s) will be provided by the Office upon request and payment of the necessary fee.

The foregoing aspects and many of the attendant advantages of this invention will become more readily appreciated and better understood by reference to the following figures.

FIG. 1: 2-Pyridine Thione (2-PT) absorbance of reduced nanoparticle-bound SPDP molecules indicating about 26 PEG chains per nanoparticle.

FIG. 2: Fluorescence of AF647-conjugated nanoparticles mapped onto a standard curve of AF647 dilutions mixed with PEG-coated nanoparticles.

FIGS. 3A-3C: Nanoparticle synthesis and characterization. FIG. 3A: Schematic illustration of synthesis of NP-PEG-MHC-AF647. Iron oxide nanoparticles were coated with a functionalized PEG to which neutravidin was covalently bound via a thioether linkage. Biotinylated peptide-MHC was attached to the PEG termini, lending the particle targeting specificity for CTLs. Neutravidin was pre-labeled with the fluorophore, Alexa Fluor® 647. FIG. 3B: Surface modification of nanoparticles with PEG and MHC/peptide verified by FTIR. FIG. 3C: Hydrodynamic size and zeta-potential of nanoparticle constructs at physiologic pH.

FIGS. 4A-4F: Targeting specificity of NP-PEG-MHC-AF647 for CTLs. Flow cytometry profile of splenocyte cell populations with targeted CTLs (FIG. 4A) or splenocytes with non-targeted CTLs (FIG. 4B) incubated with nanoparticles bearing an Alexa Fluor® 647 fluorochrome (x-axis) and stained by a FITC-labeled anti-CD8⁺ antibody (y-axis). FIG. 4C: MRI phantom image of CTL and non-CTL cells incubated with targeting nanoparticles. FIG. 4D: Flow cytometry analysis of CTLs incubated with targeting nanoparticles or peptide-MHC tetramers. FIGS. 4E and 4F: Flow cytometry analysis of CTLs⁺ incubated with targeting and non-targeting nanoparticles at two different incubation times.

FIGS. 5A and 5B: Micrographs of targeting nanoparticle-labeled CTLs. FIG. 5A: Fluorescently-labeled CTLs incubated with nanoparticles coupled with Alexa fluorophore (red, but here a medium gray, such as in the “Nanoparticles” box). The cells were labeled with a DAPI for nuclear stain (blue, but here a dark gray, such as in the “Nuclear Stain” box) and with a FITC-CD8⁺ antibody for CTL identification (green, but here a light gray, such as in the “CTL Stain” box). FIG. 5B: TEM micrograph of CTLs labeled with targeting nanoparticles. Nanoparticles are shown, bound at the surface of the T cell cross sections.

FIGS. 6A and 6B: Fluorescence (FIG. 6A) and electron microscopy (FIG. 6B) analysis of CTLs incubated with neutravidin-conjugated control nanoparticles (NP-PEG-AF647). CTLs showed little or no nanoparticle binding.

FIG. 7: Functionality of nanoparticle-labeled CTLs. Flow cytometry analysis of the functionality of CTLs incubated with control/targeting nanoparticles (left) and control nanoparticles/tetramer (right) 18 hrs post incubation. Cells incubated with nanoparticles or tetramers were probed for upregulation of CD69, an early indicator of T cell activation. Targeting nanoparticles demonstrated T cell functionality comparable to MHC-peptide tetramers after loading.

### DETAILED DESCRIPTION

Selective cell labeling offers clinicians and researchers the ability to identify specific cell populations and monitor their localization patterns throughout a biological system. In this regard, the present invention generally provides a nanoparticle system that may be used to label and monitor cells using, for example, magnetic resonance and/or fluorescence imaging. Methods of the present invention can, for example, allow for specific labeling of target cells with minimal non-specific labeling. Moreover, in certain embodiments, cells labeled by nanoparticles of the present invention retain full functionality subsequent to isolation. The nanoparticles presented herein may also allow for separation of labeled cells with magnetic columns, a technique that provides improved speed, reduced costs, simplified processing, and minimized physical and biological impact on labeled cells compared to other cell labeling methods.

Accordingly, in one aspect, the present invention provides a nanoparticle comprising: (a) a core comprising a magnetic material and having a surface; and (b) an antigenic peptide-major histocompatibility complex (MHC) monomer operatively linked to the surface, wherein the antigenic peptide-MHC monomer is recognized by a T cell receptor. The MHC may be MHC I or MHC II.

The magnetic material can be, for example, ferrous oxide, ferric oxide, silicon oxide, polycrystalline silicon oxide, silicon nitride, aluminum oxide, germanium oxide, zinc selenide, tin dioxide, titanium, titanium dioxide, indium tin oxide, gadolinium oxide, or stainless steel. In certain embodiments, the magnetic material is a doped nanoparticle. As used herein, a “doped nanoparticle” refers to nanoparticles whose host atoms in the crystal structure have been substituted by one or more atoms, where the diameter of the nanoparticle ranges from about 1-100 nm. The doped nanoparticle can be, for example, nickel titanium, MnFeO₄, CoFe₂O₄, CoFe₂O₄, or NiFe₂O₄.

As used herein, “operatively linked” refers to the joining of a nanoparticle core surface as described herein to an antigenic peptide-MHC monomer such that the antigenic peptide-MHC monomer may be recognized by a T cell receptor. Joining may be direct or indirect, wherein “indirect” indicates that one or more intervening moieties (e.g., a polymer (e.g., polyethylene glycol (PEG)), biotin, avidin, a thioether bond), are positioned between the nanoparticle core surface and the antigenic peptide-MHC monomer. Methods of determining whether the antigenic peptide-MHC monomer may be recognized by a T cell receptor are known in the art, and at least one method is described herein.

As used herein, an “antigenic peptide” is a peptide presented on an MHC I or II complex that is recognized by a T cell. As used herein, a “peptide” refers to two or more amino acids joined together by an amide bond. In certain embodiments, peptides comprise up to or include 50 amino acids. In certain embodiments, a peptide, such as an antigenic peptide, is at most or at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length, or any range derivable therein. In certain embodiments, the amino acid is at least 8 amino acids in length. As used herein, an “amino acid” refers to any of the 20 naturally occurring amino acids found in proteins.

Antigenic peptides are well-known in the art. Nanoparticles of the present invention may employ any antigenic peptide known in the art. An antigen may be a tumor-associated antigen, or not. An antigen may be a minor antigen. Non-limiting examples of antigenic peptides include pmel-1, HA-1 (a minor histocompatibility antigen), MART-1, gp100, NY-ESO-1, WT-1, GAD65, CMV pp65, EBNA, LMP2, HIV-gag, α-actinin-4, ARTC1, BCR-ABL, B-RAF, CASP-5, CASP-8, β-catenin, Cdc27, CDK4, CDKN2A, COA-1, dek-can fusion protein, EF2, ETV6-AML1 fusion protein, LDLR-fucosyltransferaseAS fusion protein, HLA-A2, HLA-A11, hsp70-2, KIAA0205, Mart2, Mum-1, 2, and 3, neo-PAP, myosin class I, OS-9, pm1-RARα fusion protein, PTPRK, K-ras, N-ras, Triosephosphate isomeras, Bage-1, Gage 3, 4, 5, 6, 7, GnTV, Herv-K-mel, Lage-1, Mage-A1, 2, 3, 4, 6, 10, 12, Mage-C2, NA-88, SP17, SSX-2, and TRP2-Int2, tyrosinase, TRP-1, TRP-2, MACE-1, p15(58), CEA, RAGE, SCP-1, Hom/Mel-40, PRAME, p53, H-Ras, HER-2/neu, E2A-PRL, H4-RET, IGH-IGK, MYL-RAR, Epstein Barr virus antigens, human papillomavirus (HPV) antigens E6 and E7, TSP-180, p185erbB2, p180erbB-3, c-met, nm-23H1, PSA, TAG-72-4, CA 19-9, CA 72-4, CAM 17.1, NuMa, p16, TAGE, PSMA, PSCA, CT7, telomerase, 43-9F, 5T4, 791Tgp72, α-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\70K, NY-CO-1, RCAS1, SDCCAG16, TA-90 (Mac-2 binding protein\cyclophilin C-associated protein), TAAL6, TAG72, TLP, DKK1, EZH2, ALDH1A1 and TPS. Additional non-limiting examples of antigenic peptides are commonly known to one of skill in the art.

In certain embodiments, a nanoparticle of the present invention further comprises a polymer that forms a coating on the surface of the magnetic material, and an antigenic peptide-MHC monomer that is operatively linked to the polymer. In certain embodiments, the hydrodynamic size of such a nanoparticle ranges from about 5-300 nm. As used herein, “hydrodynamic size” refers to the apparent size of a molecule (e.g., nanoparticle of the present invention) based on the diffusion of the molecule through an aqueous solution. More particularly, hydrodynamic size refers the radius of a hard sphere that diffuses at the same rate as the particle under examination as measured by (DLS). The hydrodynamic radius is calculated using the particle diffusion coefficient and the Stokes-Einstein equation given below, where k is the Boltzmann constant, T is the temperature, and η is the dispersant viscosity:

$${R}_{B} = \frac{kT}{6 πη D} .$$

A single exponential or Cumulant fit of the correlation curve is the fitting procedure recommended by the International Standards Organization (ISO). The hydrodynamic size extracted using this method is an intensity weighted average called the Z average. The hydrodynamic size of a nanoparticle may be about, at most about, or at least about 5, 10, 20, 30, 40, 50, 60, 70, 80, 90, 100, 110, 120, 130, 140, 150, 160, 170, 180, 190, 200, 210, 220, 230, 240, 250, 260, 270, 280, 290, or 300 nm, or any range derivable therein. In certain embodiments, the polymer that forms a coating on the surface is covalently bound to the surface, such as through a thioether linkage or an ether linkage. In other embodiments, the polymer that forms a coating on the surface is not covalently bound to the surface. For example, the polymer can be physically adsorbed to the surface.

A variety of polymers may be employed with nanoparticles of the claimed invention. Generally, any polymer may be used provided it does not produce toxic or other untoward effects in the environment or subject in which it comes into contact. Non-limiting examples of polymers that may be employed include poly(ethylene glycol) (PEG), chitosan, and chitosan-PEG. In certain embodiments, the polymer is PEG. The molecular weight of the PEG ranges from about 200-20,000 Da, for example. In certain embodiments, the molecular weight of the PEG is about, at most about, or at least about 200, 500, 750, 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, or 20,000 Da, or any range derivable therein. In certain embodiments, the molecular weight of chitosan ranges between about 100-600 Da. The molecular weight of chitosan may be about, at most about, or at least about 100, 200, 300, 400, 500, or 600 Da, or any range derivable therein. The degree of deacetylation of chitosan may range from about, at most about, or at least about 5%, 10%, 15%, 20%, 25%, 30%, 35%, 40%, 45%, 50%, 55%, 60%, 65%, 70%, 75%, 80%, 85%, 90%, 95%, 99%, or 100%, or any range derivable therein. In certain embodiments, the degree of deacetylation is greater than 50%. In certain embodiments, the degree of deacetylation is greater than 75%. In certain embodiments, the degree of deacetylation ranges between about 75-85%.

Polymers employed in embodiments of the present invention generally comprise a reactive functional group to allow for attachment to the MHC. Typically, the functional group is a nucleophile. As used herein, the term “nucleophile” or “nucleophilic” generally refers to atoms bearing lone pairs of electrons. Such terms are well known in the art and include, for example, amino (—NH₂), thiolate, sulfhydryl (—SH), and hydroxyl (—OH).

In certain embodiments, the polymer is covalently bound to an avidin protein. In certain embodiments, an antigenic peptide-MHC monomer comprises biotin and is bound to an avidin protein through a biotin/avidin interaction. Such interactions are well-known in the art. The avidin protein may be any avidin protein known in the art, such as neutravidin or streptavidin. Avidin is directly bound to the magnetic material of a nanoparticle without the use of a polymer, in certain embodiments.

Any nanoparticle of the claimed invention may further comprise a signal-generating label. Such labels may be used for detection purposes, such as for tracking or quantification. Any signal-generating label known in the art may be employed provided it does not interfere with the function of the nanoparticle, such as its targeting ability or its stability. Non-limiting examples of signal-generating labels include fluorophores, chromophores, and radiolabels. In certain embodiments, the signal-generating label is a fluorophore, such as a near-infrared fluorophore (NIRF) or a visible light fluorophore. Non-limiting examples of near-infrared fluorophores include the cyanines (e.g., Cy5.5), Alexa Fluors® (e.g., Alexa Fluor® 680), or DyLights™ (e.g., DyLight™ 680). In certain embodiments, a visible light fluorophore is employed for in vitro applications.

The signal-generating label(s) may be attached to any component of the nanoparticle, such as to the magnetic material of the nanoparticle, a polymer, an avidin protein, a MHC monomer, or an antigenic peptide, or any combination thereof. In certain embodiments, a nanoparticle comprises a polymer that forms a coating on the surface, and an antigenic peptide-MHC monomer is operatively linked to the polymer, and the polymer is further covalently bound to a fluorophore-labeled avidin protein.

Any cell that comprises a T cell receptor may be employed in aspects of the present invention. In certain embodiments, a normal cell (e.g., a cell that is not a tumor cell) comprises the T cell receptor. Persons of skill in the art are familiar with such cells. Non-limiting examples of such cells include pancreatic islet cells and helper T cells. Cells involved in autoimmune diseases may comprise a T cell receptor. Also, NKT cells may comprise a T cell receptor for purposes of the present invention.

In certain embodiments, a tumor cell-specific CD4 or CD8 T cell comprises the T cell receptor. Non-limiting examples of types of CD4 cells include helper T cells, regulatory T cells (T_{regs}), TH1, TH2, TH and TH17 cells. Non-limiting examples of CD8 cells include T-suppressor cells and cytotoxic T lymphocytes.

The tumor may be of any type known in the art. In certain embodiments, the tumor cell is selected from the group consisting of a melanoma cell, a chronic myelogenous leukemia (CML) cell, an acute myeloid leukemia (AML) cell, a breast cell, a lung cell, a brain cell, a liver cell, a pancreas cell, a prostate cell, a lymphoma cell, an ovarian cell, a uterine cell, a stomach cell, a colon cell, a kidney cell, an esophageal cell, a testicular cell, a bone cell, a thyroid cell, a cardiac cell, a cervical cell, a skin cell, a urinary tract cell, a bladder cell and a mouth cell. In certain embodiments, the tumor cell is a melanoma cell. In particular embodiments, the tumor cell is a melanoma cell and the antigenic peptide is pmel-1.

Nanoparticles of the present invention may have a mean core size (that is, mean diameter of the core) of about 5-12 nm. In certain embodiments, the mean core size is about, at most about, or at least about 5, 6, 7, 8, 9, 10, 11, or 12 nm, or any range derivable therein. In certain embodiments, the mean core size is about 10 nm.

Other embodiments of the present invention include a nanoparticle, comprising: (a) a core comprising a magnetic material and having a surface coated with a polymer; and (b) an antigenic peptide-major histocompatibility complex (MHC) monomer operatively linked to the polymer, wherein the antigenic peptide-MHC monomer is recognized by a T cell receptor.

Nanoparticles of the present invention also include, for example, a nanoparticle comprising: (a) a core comprising a magnetic material and having a surface covalently bound to a polymer; (b) a biotinylated antigenic peptide-major histocompatibility complex (MHC) monomer operatively linked to the polymer, wherein the antigenic peptide-MHC monomer is recognized by a T cell receptor; and (c) an avidin protein that is bound to the antigenic peptide-MHC monomer through a biotin/avidin interaction and is also covalently bound to the polymer.

In certain embodiments, a nanoparticle comprises: (a) a core comprising a magnetic material and having a surface; (b) an antigenic peptide-major histocompatibility complex (MHC) monomer operatively linked to the surface, wherein the antigenic peptide-MHC monomer is recognized by a T cell receptor; and (c) a fluorophore.

In certain aspects, a nanoparticle comprises: (a) a core comprising a magnetic material and having a surface covalently bound to a polymer; (b) an antigenic peptide-major histocompatibility complex (MHC) monomer operatively linked to the polymer, wherein the antigenic peptide-MHC monomer is recognized by a T cell receptor; (c) an avidin protein operatively linked to the antigenic peptide-MHC monomer and also covalently bound to the polymer; and (d) a fluorophore.

Nanoparticles of the present invention may also be comprised in a composition, wherein the composition comprises a pharmaceutically acceptable carrier, excipient or diluent, suitable for administration to a subject. Such carriers are described herein along with methods of administration to a subject.

A method of the present invention, in certain embodiments, comprises: detecting the presence of cells having a T cell receptor in a sample, comprising: (a) contacting the sample with a nanoparticle comprising: (i) a core comprising a magnetic material and having a surface; and (ii) an antigenic peptide-major histocompatibility complex (MHC) monomer operatively linked to the surface, wherein the antigenic peptide-MHC monomer is recognized by the T cell receptor; and (b) measuring the level of nanoparticle binding to cells in the sample using magnetic resonance imaging. At least one method of measurement using magnetic resonance imaging is described herein. In certain embodiments, the cells are further defined as tumor cell-specific cytotoxic T cells. The cells in this or any other method may be in vitro or ex vivo. The sample in this or any other method can be, for example, a tissue. This or any other method discussed herein may further comprise isolating those cells that bound to the nanoparticle.

A method of the present invention, in certain embodiments, comprises detecting the presence of cells having a T cell receptor in a subject, comprising: (a) administering to the subject a nanoparticle comprising: (i) a core comprising a magnetic material and having a surface; and (ii) an antigenic peptide-major histocompatibility complex (MHC) monomer operatively linked to the surface, wherein the antigenic peptide-MHC monomer is recognized by the T cell receptor; and (b) measuring the level of nanoparticle binding to cells in the subject using magnetic resonance imaging. Methods of magnetic resonance imaging in subjects are well-known in the art.

Administration of nanoparticles of the present invention in this or any other method described herein regarding a subject is by, for example, injection, such as intravenous injection or intratumoral injection. Other methods of administration are discussed herein.

As used herein, the term “patient” or “subject” refers to a living mammalian organism, such as a human, monkey, cow, sheep, goat, dog, cat, rabbit, mouse, rat, guinea pig, or transgenic species thereof. In certain embodiments, the patient or subject is a primate. Non-limiting examples of human subjects are adults, juveniles, infants, and fetuses.

The present invention also contemplates methods drawn to adoptive cell transfer (ACT). Accordingly, in certain aspects of the present invention, methods comprise detecting the presence of T cells having a T cell receptor in a subject through steps including: (a) removing T cells from a subject; (b) performing expansion of the T cells; (c) contacting the expanded T cells with a nanoparticle comprising: (i) a core comprising a magnetic material and having a surface; and (ii) an antigenic peptide-major histocompatibility complex (MHC) monomer operatively linked to the surface, wherein the antigenic peptide-MHC monomer is recognized by the T cell receptor; (d) introducing the expanded T cells that have been contacted with a nanoparticle as in step (c) back into the subject; and (e) measuring the level of nanoparticle binding to the T cells in the subject using magnetic resonance imaging.

Other methods of detection of cells having a T cell receptor in a sample may comprise fluorescence detection. For example, such a method can comprise: (a) contacting the sample with a nanoparticle comprising: (i) a core comprising a magnetic material and having a surface; (ii) an antigenic peptide-major histocompatibility complex (MHC) monomer operatively linked to the surface, wherein the antigenic peptide-MHC monomer is recognized by the T cell receptor; and (iii) a fluorophore; (b) isolating those cells from the sample that bound to a nanoparticle; and (c) measuring the level of nanoparticle binding to cells in the sample using fluorescence detection.

In certain embodiments, a method of the present invention comprises a method of determining the localization of a nanoparticle in a cell, comprising: (a) contacting the cell with a nanoparticle, wherein the nanoparticle comprises: (i) a core comprising a magnetic material and having a surface; (ii) an antigenic peptide-major histocompatibility complex (MHC) monomer operatively linked to the surface, wherein the antigenic peptide-MHC monomer is recognized by a T cell receptor; and (iii) a fluorophore; and (b) detecting the location of the nanoparticle in the cell using fluorescence detection. Such methods may further comprise isolating the cell that bound to the nanoparticle.

Methods of the present invention may comprise methods of making the nanoparticles described herein. For example, a method of making a nanoparticle can comprise: (a) obtaining a core comprising a magnetic material and having a surface; (b) coating the surface with a polymer; (c) covalently coupling an avidin protein to the polymer to form a core-polymer-avidin protein complex; (d) biotinylating an antigenic peptide-major histocompatibility complex (MHC) monomer; and (e) coupling the core-polymer-avidin protein complex to the biotinylated antigenic peptide-MHC monomer. As noted above, any component of a nanoparticle of the present invention may comprise a signal-generating label, such as a fluorophore. For example, in a method of making a nanoparticle, an avidin protein is labeled with a fluorophore.

The use of the term “or” in the claims is used to mean “and/or” unless explicitly indicated to refer to alternatives only or the alternatives are mutually exclusive. It is specifically contemplated that any listing of items using the term “or” means that any of those listed items may also be specifically excluded from the related embodiment.

Throughout this application, the term “about” is used to indicate that a value includes the standard deviation of error for the device or method being employed to determine the value.

As used herein the specification, “a” or “an” may mean one or more, unless clearly indicated otherwise. As used herein in the claims, when used in conjunction with the word “comprising,” the words “a” or “an” may mean one or more than one.

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has” or “includes” one or more steps is not limited to possessing only those one or more steps and also covers other unlisted steps.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. For example, any method discussed herein may employ any nanoparticle described herein.

### Pharmaceutical Formulations and Administration

Pharmaceutical compositions of the present invention comprise an effective amount of one or more candidate substances (e.g., a nanoparticle of the present invention) or additional agents dissolved or dispersed in a pharmaceutically acceptable carrier. As used herein, the term “effective” (e.g., “an effective amount”) means adequate to accomplish a desired, expected, or intended result. The phrases “pharmaceutical or pharmacologically acceptable” refers to molecular entities and compositions that do not produce an adverse, allergic, or other untoward reaction when administered to an animal, such as, for example, a human, as appropriate.

Guidelines for the preparation of a pharmaceutical composition that contains at least one candidate substance or additional active ingredient, such as a pharmaceutically acceptable carrier, may be provided in light of the present disclosure and through consultation of Remington's Pharmaceutical Sciences, 18th Ed. Mack Printing Company, 1990. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials, and combinations thereof as would be known to one of ordinary skill in the art (see, for example, Remington's Pharmaceutical Sciences, pp 1289-1329, 1990). Except insofar as any conventional carrier is incompatible with the active ingredient, its use in pharmaceutical compositions is contemplated.

The candidate substance may comprise different types of carriers depending on whether it is to be administered in solid, liquid, or aerosol form, and whether it needs to be sterile for such routes of administration as injection. Nanoparticles of the present invention may be administered orally, intraadiposally, intraarterially, intraarticularly, intracranially, intradermally, intralesionally, intramuscularly, intranasally, intraocularally, intrapericardially, intraperitoneally, intrapleurally, intraprostaticaly, intrarectally, intrathecally, intratracheally, intraumbilically, intravaginally, intravenously, intravesicularlly, intravitreally, liposomally, locally, mucosally, orally, parenterally, rectally, subconjunctival, subcutaneously, sublingually, topically, transbuccally, transdermally, vaginally, in crémes, in lipid compositions, via a catheter, via a lavage, via continuous infusion, via infusion, via inhalation, via injection, via local delivery, via localized perfusion, bathing target cells directly, or by other method or any combination of the foregoing. In particular embodiments, the composition is formulated for delivery via injection, such as intravenous or intratumoral injection. Pharmaceutical compositions comprising nanoparticles of the present invention may be adapted for administration via any method known to those of skill in the art, such as the methods described above.

In particular embodiments, the composition is administered to a subject using a drug delivery device. Any drug delivery device is contemplated for use in delivering a pharmaceutically effective amount of a nanoparticle of the present invention.

The actual dosage amount of a nanoparticle as described herein administered to a subject may be determined by physical and physiological factors such as body weight, severity of condition, the type of disease being detected, or monitored, previous or concurrent therapeutic interventions, idiopathy of the patient, and on the route of administration. The practitioner responsible for administration will typically determine the concentration of active ingredient(s) in a composition and appropriate dose(s) for the individual subject. Nanoparticles of the present invention may be cleared by the kidneys; thus, it may be important to assess any underlying problems with kidney function. Kidney function may be assessed by measuring the blood levels of creatinine, a protein normally found in the body. If these levels are higher than normal, it is an indication that the kidneys may not be functioning at an optimal rate and dosage may be lowered accordingly.

The dose may be repeated as needed as determined by those of ordinary skill in the art. Thus, in some embodiments of the methods set forth herein, a single dose is contemplated. In other embodiments, two or more doses are contemplated. Where more than one dose is administered to a subject, the time interval between doses can be any time interval as determined by those of ordinary skill in the art. For example, the time interval between doses may be about 5-30 minutes, about 0.5-1 hour, about 1-2 hours, about 2-6 hours, about 6-10 hours, about 10-24 hours, about 1-2 days, about 1-2 weeks, or longer, or any time interval derivable within any of these recited ranges.

In certain embodiments, it is desirable to provide a continuous supply of a pharmaceutical composition to the patient. This could be accomplished by continuous injection, for example.

In certain embodiments, pharmaceutical compositions comprise, for example, at least about 0.1% of a nanoparticle as described herein. In other embodiments, a nanoparticle comprises between about 2% to about 75% of the weight of the unit, or between about 25% to about 60%, for example, and any range derivable therein. In other non-limiting examples, a dose comprises from about, at most about, or at least about 1, 5, 10, 50, or 100 microgram/kg/body weight, 1, 5, 10, 50, or 100 milligram/kg/body weight, or 1000 mg/kg/body weight or more per administration, or any range derivable therein. In non-limiting examples of a derivable range from the numbers listed herein, a range of about 5 mg/kg/body weight to about 100 mg/kg/body weight or about 5 microgram/kg/body weight to about 500 milligram/kg/body weight can be administered.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal, or combinations thereof.

The nanoparticles described herein may be formulated into a composition, such as a pharmaceutical composition, in a free base, neutral, or salt form. Compositions comprising pharmaceutically acceptable salts are therefore contemplated. The term “pharmaceutically acceptable salts” as used herein refers to salts of nanoparticles of this invention that are substantially non-toxic to living organisms. Typical pharmaceutically acceptable salts include those salts prepared by reaction of a nanoparticle of this invention with an inorganic or organic acid or an organic base, depending on the substituents present on the compounds of the invention.

Non-limiting examples of inorganic acids that may be used to prepare pharmaceutically acceptable salts include: hydrochloric acid, phosphoric acid, sulfuric acid, hydrobromic acid, hydroiodic acid, phosphorous acid, and the like. Non-limiting examples of organic acids that may be used to prepare pharmaceutically acceptable salts include: aliphatic mono- and dicarboxylic acids, such as oxalic acid, carbonic acid, citric acid, succinic acid, phenyl-heteroatom-substituted alkanoic acids, aliphatic and aromatic sulfuric acids, and the like. Pharmaceutically acceptable salts prepared from inorganic or organic acids thus include hydrochloride, hydrobromide, nitrate, sulfate, pyrosulfate, bisulfate, sulfite, bisulfate, phosphate, monohydrogenphosphate, dihydrogenphosphate, metaphosphate, pyrophosphate, hydroiodide, hydrofluoride, acetate, propionate, formate, oxalate, citrate, lactate, p-toluenesulfonate, methanesulfonate, maleate, and the like.

Pharmaceutically acceptable salts also include the salts formed between carboxylate or sulfonate groups found on some of the nanoparticles of this invention and inorganic cations, such as sodium, potassium, ammonium, or calcium, or organic cations such as isopropylammonium, trimethylammonium, tetramethylammonium, and imidazolium. Suitable pharmaceutically acceptable salts may also be formed by reacting the agents of the invention with an organic base such as methylamine, ethylamine, ethanolamine, lysine, ornithine, and the like.

It should be recognized that the particular anion or cation forming a part of any salt of this invention is typically not critical, so long as the salt, as a whole, is pharmacologically acceptable. Additional examples of pharmaceutically acceptable salts and their methods of preparation and use are presented in Handbook of Pharmaceutical Salts: Properties, Selection and Use (P. H. Stahl & C. G. Wermuth eds., Verlag Helvetica Chimica Acta, 2002).

In embodiments where the composition is in a liquid form, a carrier may be a solvent or dispersion medium comprising, but not limited to, water, ethanol, polyol (e.g., glycerol, propylene glycol, liquid polyethylene glycol), lipids (e.g., triglycerides, vegetable oils, liposomes), and combinations thereof. The proper fluidity can be maintained, for example, by the use of a coating, such as lecithin; by the maintenance of the required particle size by dispersion in carriers such as, for example, liquid polyol or lipids; by the use of surfactants such as, for example, hydroxypropylcellulose; or combinations thereof such methods. It may be preferable to include isotonic agents, such as, for example, sugars, sodium chloride, or combinations thereof.

Sterile injectable solutions may be prepared by incorporating a nanoparticle of the present invention in the required amount in the appropriate solvent with various of the other ingredients enumerated above, as required, followed by filtered sterilization. Generally, dispersions are prepared by incorporating the various sterilized active ingredients into a sterile vehicle that contains the basic dispersion medium and/or the other ingredients. In the case of sterile powders for the preparation of sterile injectable solutions, suspensions or emulsion, certain methods of preparation may include vacuum-drying or freeze-drying techniques that yield a powder of the active ingredient plus any additional desired ingredient from a previously sterile-filtered liquid medium thereof. The liquid medium should be suitably buffered if necessary and the liquid diluent (e.g., water) first rendered isotonic prior to injection with sufficient saline or glucose. The preparation of highly concentrated compositions for direct injection is also contemplated, where the use of DMSO as solvent is envisioned to result in extremely rapid penetration delivering high concentrations of the active agents to a small area.

The composition should be stable under the conditions of manufacture and storage and preserved against the contaminating action of microorganisms, such as bacteria and fungi. It will be appreciated that endotoxin contamination should be kept minimally at a safe level, for example, less that 0.5 ng/mg protein.

While illustrative embodiments have been illustrated and described, it will be appreciated that various changes can be made therein without departing from the spirit and scope of the invention.

The following examples are provided for the purpose of illustrating, not limiting, the invention.

All chemicals were purchased from Sigma Chemicals (St. Louis, Mo.) unless otherwise specified.

### EXAMPLES

### Example 1

### Preparation and Characterization of Magnetic Nanoparticles Comprising Multiple Polymers (NP-PEG)

Preparation of Magnetic Nanoparticles. Magnetite iron oxide nanoparticles were prepared by adding a 1.5M sodium hydroxide solution to a mixture of ferric chloride (50.9 mg/mL) and ferrous chloride tetrahydrate (30.9 mg/mL) dissolved in 0.12M hydrochloric acid under mechanical stirring and ultrasonication to shift the final pH of the solution to 12. The resulting black precipitate was isolated with a rare-earth magnet and washed with deionized water until a pH of 10.5 was reached. Following adjustment of the pH of the colloidal solution to pH 9.0 by addition of 1M hydrochloric acid, the solution was filtered through 0.65 μm cellulose membranes (Millipore, Billerica, Mass.).

Preparation of a Heterobifunctional PEG Chain. The surface of the nanoparticle was modified with a heterobifunctional poly(ethylene glycol) chain (MW 600). First, in a round-bottom flask, 100 g (0.167 mol) of PEG biscarboxylate were degassed under a 2-Torr vacuum to remove residual water and air in the liquid. Following degassing, 35 mL (0.48 mol) of thionyl chloride were added dropwise to the neat PEG, converting it to the corresponding diacid chloride Initially, vigorous bubbling was observed followed by slow bubbling resulting from HCl and SO₂ gas. The sample was heated for 1.5 h under nitrogen, followed by degassing under a 2-Torr vacuum to remove SO₂ gas and excess thionyl chloride.

Next, 32 mL (0.44 mol) of 2,2,2-trifluoroethanol were added dropwise to the PEG diacid chloride under nitrogen. The solution was stirred for 2 h and heated to reflux for 3 h After being cooled to room temperature, the resulting mixture was placed under a 2-Torr vacuum to remove residual trifluoroethanol.

To prepare the PEG silane, 20 g of PEG-ditrifluoroethylester were dissolved in 200 mL of dry toluene. The solution was heated to reflux under nitrogen utilizing a Dean-Stark apparatus to remove residual water from the solution. When the resultant solution cooled to room temperature, 6.4 mL (27 mmol) or APS were added dropwise to the PEG-ditrifluoroethylester solution under nitrogen. The resultant solution was stirred overnight under nitrogen at ambient temperature. Following the amidation reaction, the solvent was removed by distillation. After the distillation, a 0.2 Torr vacuum was applied to remove residual toluene and APS from the PEG silane to yield the crude half amide-ester.

Modification of Magnetic Nanoparticles with a Heterobifunctional PEG. Magnetic nanoparticles (200 mg) were dispersed in 100 mL of toluene in a round-bottom flask by 20 min of sonication. Following dispersion, 1 mL, of the PEG-trifluoroethylester was added to the nanoparticle suspension, and the mixture was sonicated for 4 h at 50° C. The resultant PEG-immobilized nanoparticle precipitate was isolated by centrifugation and washed three times with dry toluene to remove residual PEG-silane. The primary mine was created on the immobilized PEG chain termini by flooding the nanoparticle suspension with excess ethylenediamine (EDA). Next, 1 mL of EDA was added to the PEG immobilized nanoparticle suspension and allowed to react for 2 h. The particles were then isolated with a rare earth magnet and washed three times with deionized (Dl) water.

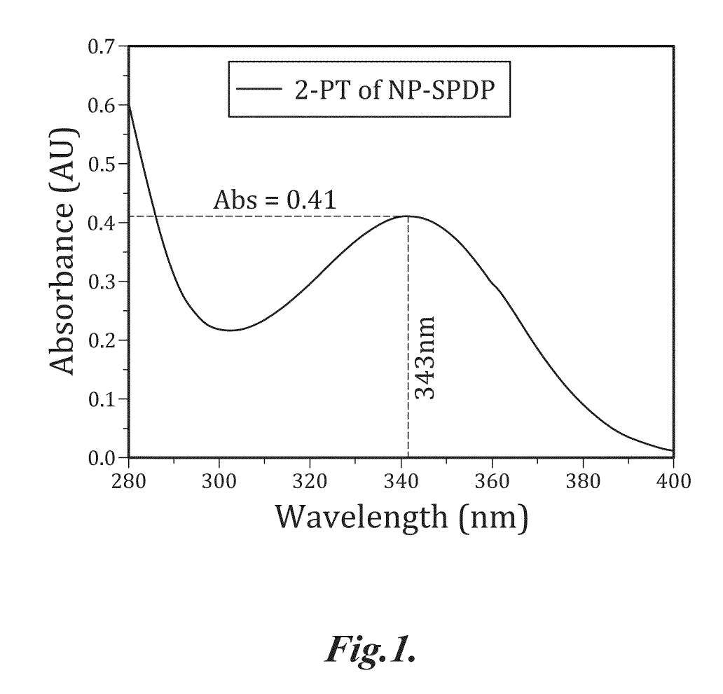

Quantitation. Conjugation of an N-succinimidyl-3-(2-pyridyldithio)-propionate (SPDP) linker molecule to the amine-terminated nanoparticles of the previous step allowed for quantitation of the activated groups after treatment with a reducing agent (dithiothreitol). Each bound SPDP group releases a cyclical 2-pyridine thione (2-PT) group with an absorption at 343 nm. UV/Vis analysis of separated 2-PT groups from a nanoparticle stock of known concentration (FIG. 1) yielded an estimated 26.26 terminal-active PEG chains per nanoparticle.

### Example 2

### Preparation and Characterization of a Fluorophore-Labeled Avidin Protein (Neutravidin-AF647)

Neutravidin (10 mg; Molecular Probes, Eugene, Oreg.) was dissolved in 1 mL PBS and reacted with 43 μL Alexa Fluor® 647 monosuccinimidyl ester (10 mg/mL in anhydrous DMSO; Molecular Probes). The mixture was placed on a shaker and reacted at room temperature for 1 hr. Unreacted dye was removed with a PD-10 desalting column equilibrated with 50 mM Na Bicarbonate pH 8.5.

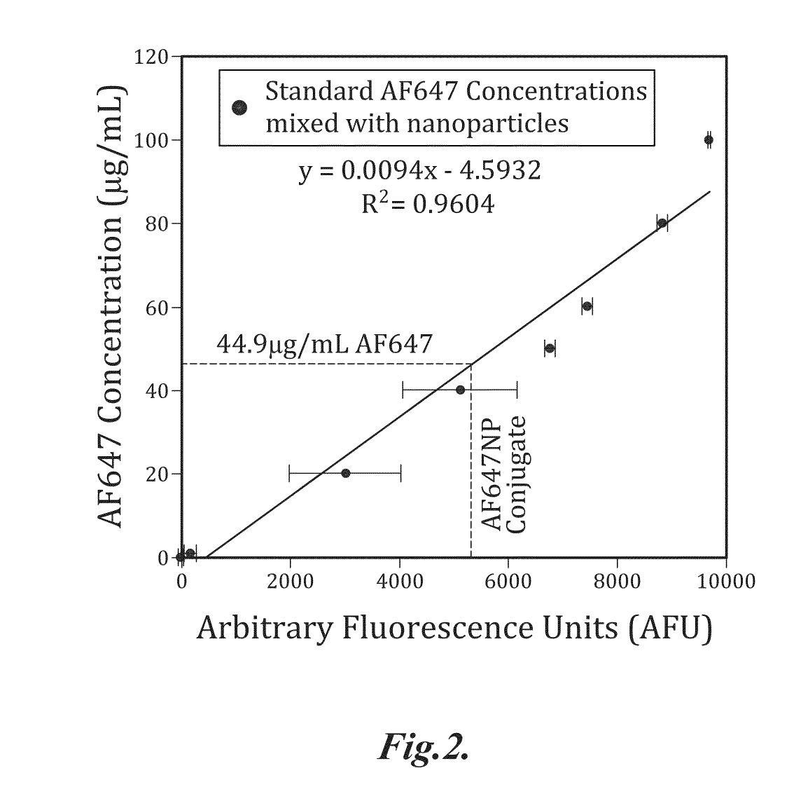

Fluorescence quantitation of labeled neutravidin yielded an estimated 2.59 AF647 dyes per protein. Subsequent analysis of neutravidin-AF647 modified nanoparticle fluorescence against AF647 dilutions in a nanoparticle mixture (common nanoparticle concentration of 50 μg Fe/mL) gave an estimated fluorophore concentration of 44.9 μg/mL, corresponding to 13.0 neutravidins per nanoparticle (FIG. 2). As each neutravidin protein has four biotin binding sites, conjugated nanoparticles retain an estimated 52 biotin binding sites for conjugation with biotinylated peptide-MHC targeting complexes.

### Example 3

### Preparation of a Multihistocompatibility Complex Coupled to a Peptide (Peptide-MHC)

Peptide-MHC monomers were used to impart targeting specificity to the NP-PEG. Melanoma-reactive CTLs specific for the gp100₂₅-33 epitope restricted by H-2D(b) (called pmel-1) can be tagged using multimers of the peptide-MHC complex presenting the pmel-1 peptide. Pmel-1 peptide MHC monomers were synthesized and biotinylated.

### Example 4

### Preparation and Characterization of NP-PEG-MHC-AF647 Nanoparticles

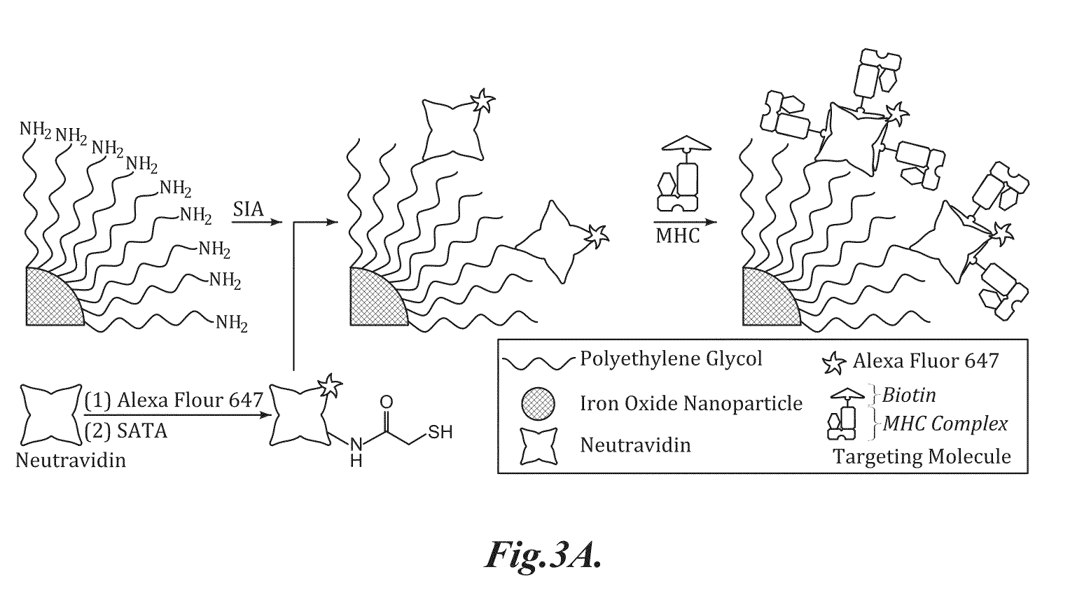

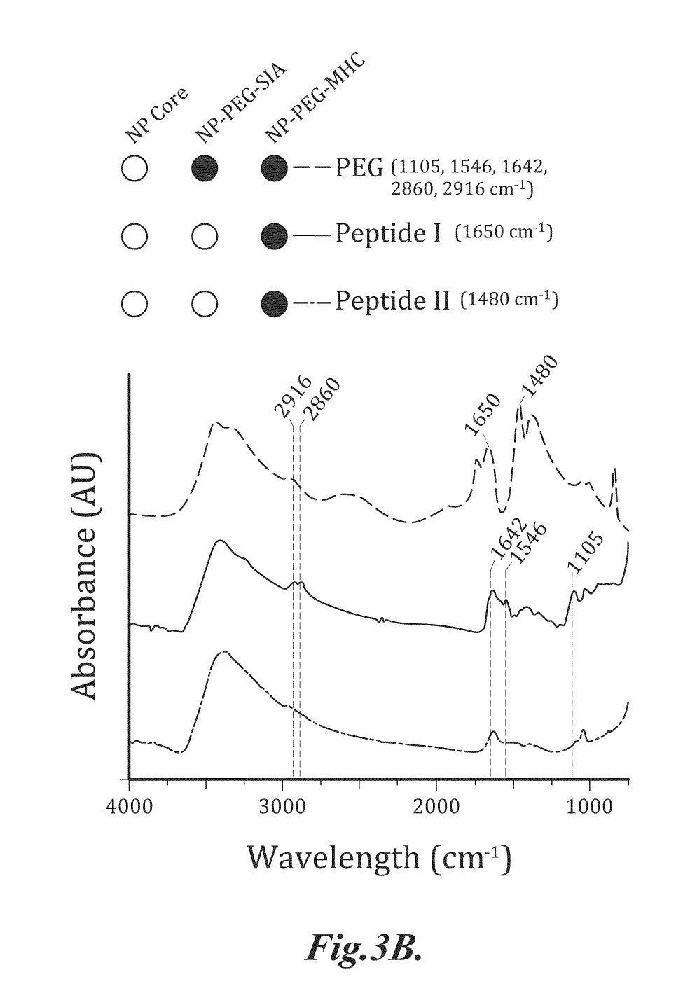

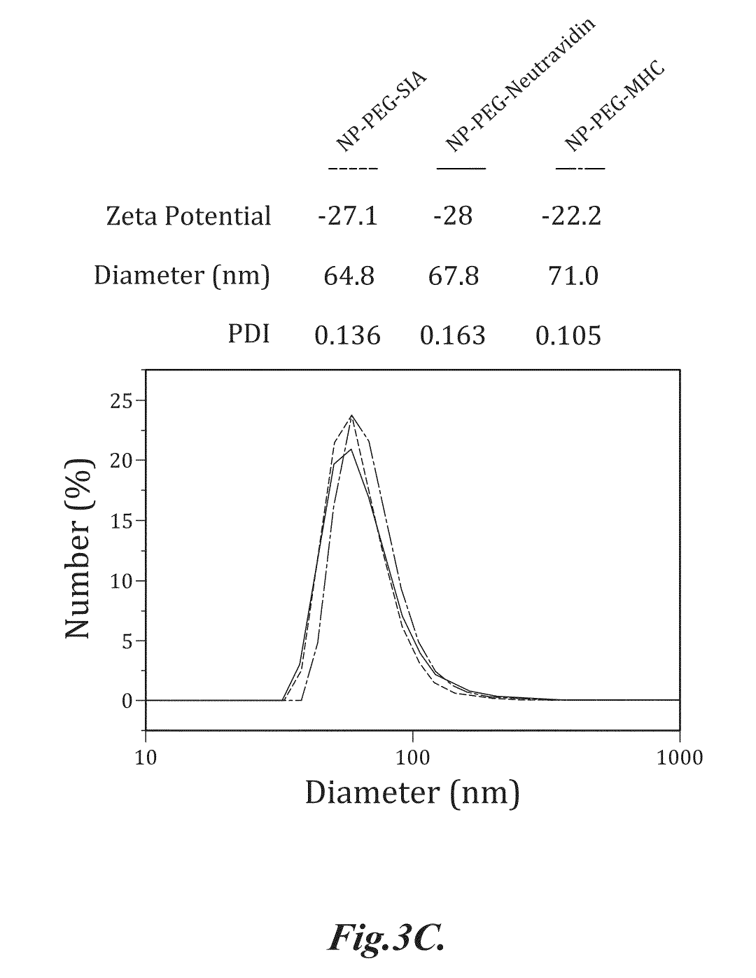

The fluorophore-labeled neutravidin protein of Example 2 (neutravidin-AF647), with strong affinity for biotinylated peptide-major histocompatibility complex (MHC), was coupled to the NP-PEG of Example 1 through a three-step process as illustrated in FIG. 3A.

Step 1: NP-PEG were isolated on a rare-earth magnet and washed twice in 150 mM boric acid pH 8.0. To 2 g Fe nanoparticles (1 mg/mL) was added 200 μL of N-Succinimidyl iodoacetate (SIA; Molecular Biosciences, Boulder, Colo.; 1 mg/mL in anhydrous DMSO), and the mixture was placed on a shaker at room temperature for 2 hrs. Unreacted SIA was removed with a Sephacryl S-200 HR column (GE Healthcare, Piscataway, N.J.) against 150 mM boric acid pH 8.0.

Step 2: Fluorophore-labeled neutravidin (1 mg in 500 μL) was mixed with 11.8 μL N-Succinimidyl-S-acetylthioacetate (SATA; Molecular Biosciences; 0.6 mg/mL in anhydrous DMSO) and allowed to react for 2 hrs at room temperature. The neutravidin-SATA solution was then mixed with a deprotection solution (55 μL of 0.5 M hydroxylamine and 25 mM EDTA, pH 7.2) for 40 min at room temperature. The mixture was then passed through a Zeba™ spin column equilibrated with 100 mM boric acid pH 8. Isolated neutravidin was mixed with 2 mg Fe SIA-modified nanoparticles overnight.

Step 3: Nanoparticles were passed through a Sephacryl® S-200 HR column equilibrated against 0.1 M boric acid pH 8.0, then isolated on a rare earth magnet, and redispersed in the same buffer. Nanoparticle concentration was determined by inductively coupled plasma atomic emission spectroscopy, and 100 μg peptide-MHC from Example 3 was mixed with 288 μg Fe nanoparticles (700 μL total volume) for 30 min.

Nanoparticle coating and surface functionalization with peptide-MHC monomers was confirmed by Fourier transform infrared spectroscopy (FUR) (FIG. 3B). All analyzed nanoparticles showed a broad —OH stretch above 3000 cm⁻¹ distinctive of the iron oxide surface. PEG-silane modified nanoparticles (NP-PEG-SIA) showed characteristic carbonyl (1642 and 1546 cm¹) and methylene bands (2916 and 2860 cm¹) of the immobilized polymer, and a Si—O peak (1105 cm¹) indicating covalent binding of PEG to the nanoparticle surface. Complete nanoparticle constructs displaying the peptide-MHC monomers (NP-PEG-MHC-AF647), likewise displayed characteristic PEG peaks as well as amide I and amide II peaks (1650 and 1480 cm⁻¹, respectively) indicating the protein immobilization at the particle surface.

The hydrodynamic size of nanoparticles was measured with dynamic light scattering (DLS) using a Malvern® Nano Series ZS particle size analyzer (Worcestershire, UK). The iron concentration of nanoparticle samples was 200 μg/mL. The hydrodynamic size of the PEG-coated nanoparticle was 64.8 nm, increasing minimally to 71.0 nm (PDI 0.105) subsequent to attachment of neutravidin (NP-PEG-neutravidin) and peptide-MHC (NP-PEG-MHC-AF647; FIG. 3C). Likewise, nanoparticle zeta-potential remained consistent during particle preparation (FIG. 3C).

### Example 5

### Peptide-MHC Tetramer Preparation

MHC-tetramer-AF647, a standard labeling molecule for T cell isolation, served as a benchmark to provide a quantitative measure of the labeling efficacy of NP-PEG-MHC-AF647 (Example 4).

The peptide-MHC tetramer was synthesized as follows. Recombinant MHC Class-I heavy chain (in this case, D^{b}) and β2-microglobulin were expressed in E. coli and purified from the inclusion body. The gp100₂₅₋₃₃ pmel-1 peptide was folded into the MHC complex by dilution of the proteins, and the peptide-MHC complex purified by gel-filtration. The product was then biotinylated using the BirA enzyme (Affinity, LLC, Denver, Colo.) and re-purified by gel filtration. The tetramer was formed by mixing biotinylated peptide-MHC complex with Alexa Fluor® 647-conjugated Streptavidin (Invitrogen) at 4:1 ratio.

### Example 6

### Splenocyte Isolation

Pmel-1 is a transgenic mouse strain on a C57BL/6 background obtained from Jackson Laboratories. The transgene encodes a gp100₂₅₋₃₃-specific, H-2Db-restricted CD8+TCR. Pmel-1 mice were bred and housed at the Fred Hutchison Cancer Research Center (Seattle, Wash.) animal facilities in a specific pathogen-free environment. Splenocytes were obtained from 6-10 week old Pmel-1 mice and B6 wild-type mice, filtered by passage through a 25 g needle and incubated in RPMI 1640 with 10% heat-inactivated fetal bovine serum, 2 mM L-glutamine, 25 mM Hepes, 1 mM sodium pyruvate, 100 μg/ml streptomycin, and 100 μg/ml penicillin.

### Example 7

### Cell Binding Study with NP-PEG-MHC-AF647 Nanoparticles

To evaluate the specific binding of the nanoparticle to CTLs (T cells expressing T cell receptors (TCR)) among various types of cells present in a splenocyte population, NP-PEG-MHC-AF647 (targeting nanoparticles of Example 4), NP-PEG-AF647 (non-targeting nanoparticles, as control for comparison), and MHC-tetramer-AF647 (Example 5) were incubated for 30 min with splenocytes isolated from Pmel-1 transgenic mice (comprised of targeted CTLs expressing the T cell receptor for the melanoma-associated pmel epitope) or the splenocytes isolated from B6 transgenic mice (representing control non-targeted CTLs). The control nanoparticles were prepared as PEG-MHC-AF647, but without the MHC conjugation step.

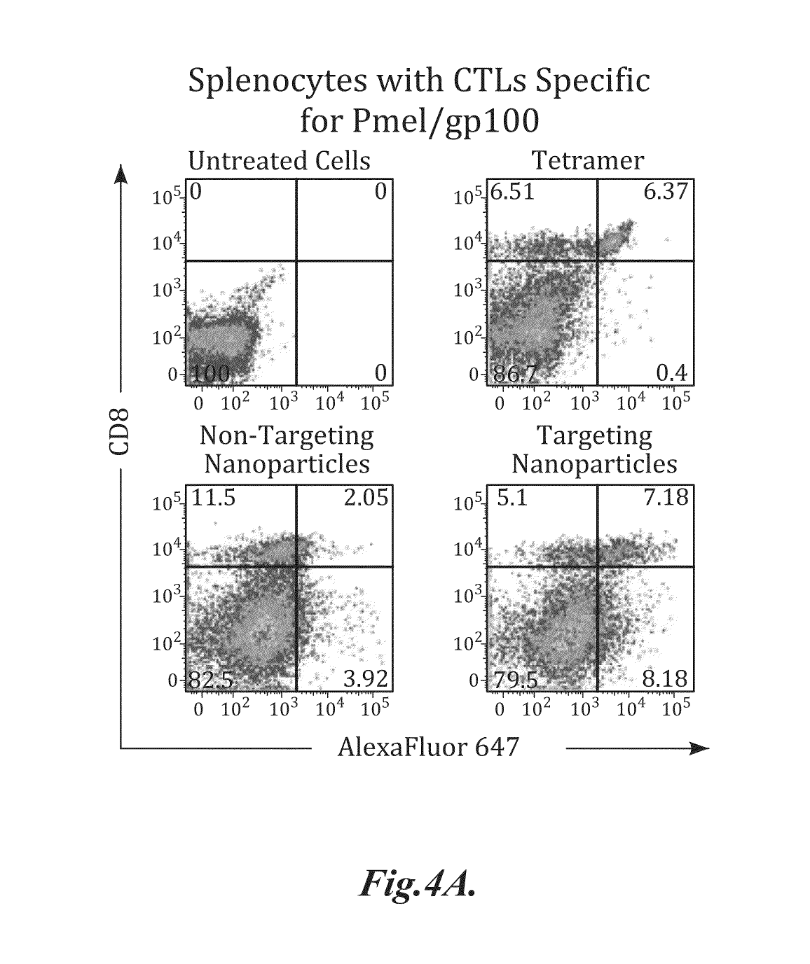

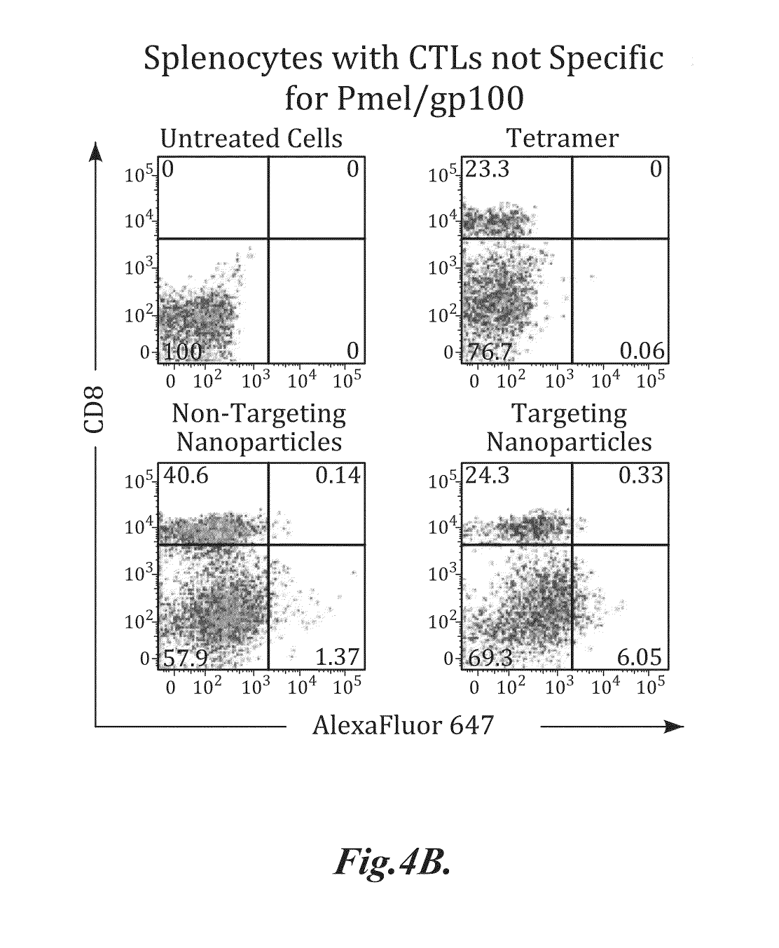

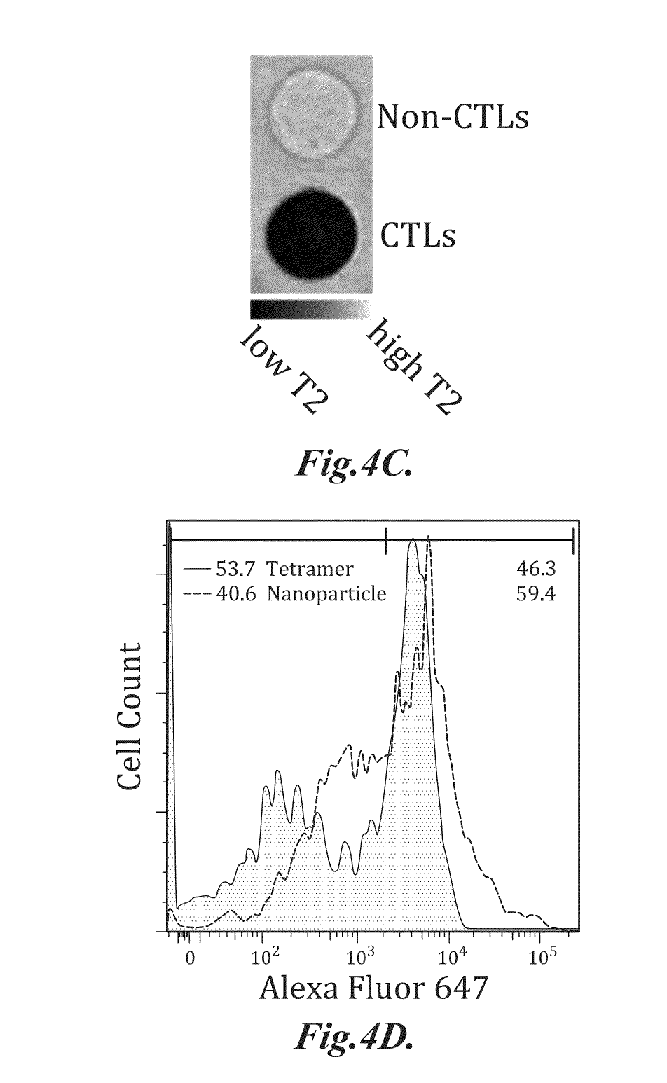

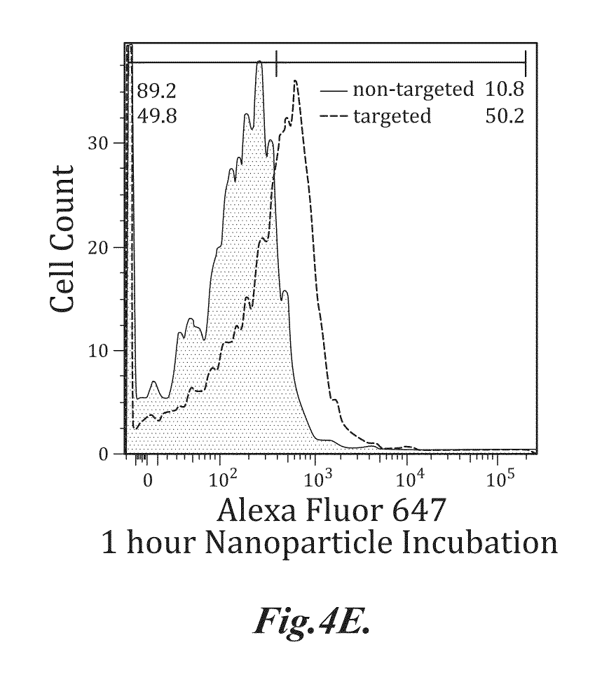

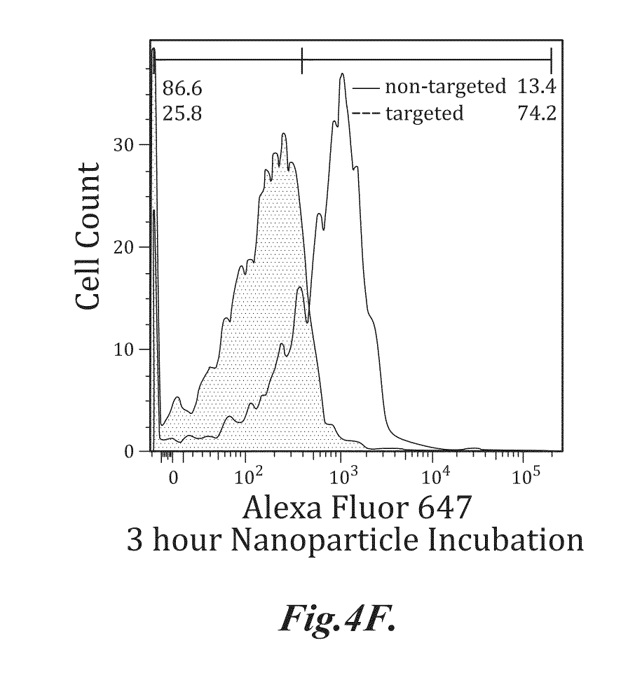

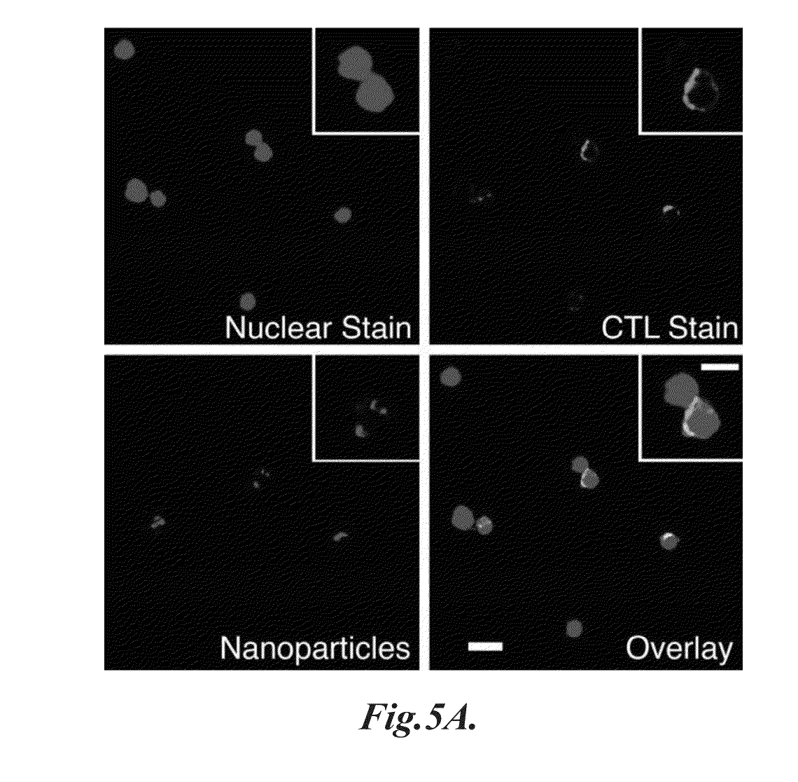

After incubation, cells were washed to remove unbound nanoparticles or tetramers, labeled with a fluorescein-isothiocyanate (FITC)-labeled anti-CD8 antibody to identify CTLs (CD8⁺), and analyzed by flow cytometry (FIGS. 4A and 4B). This protocol entailed incubation of primary cell cultures in media at ˜3×10⁶ cells/mL. Peptide-MHC labeled nanoparticles and unlabeled nanoparticles (control) were incubated with cells at 0.1 mg/mL Fe for 1 or 3 hrs. Alternatively, cells were incubated with 35 μL peptide-MHC tetramer (0.12 mg/mL) for 1 hr at 37° C. Cells were washed of unbound nanoparticles or tetramer 3× with 0.2% FBS by centrifugation and incubated with anti CD8⁺-FITC antibody for 15 min at room temperature. Cells were again washed 3× with 0.2% FBS by centrifugation. CD69 analysis was conducted 18 hr post nanoparticle/tetramer incubation. Here, cells were incubated with fluorochrome-conjugated anti-CD69 for 15 min, followed by 3× washes with 0.2% FBS. Flow cytometry analysis was performed on a BD™ LSR II; data analysis was performed with the FlowJo software package. A minimum of 10,000 cells were counted for each sample. Cell samples for transmission electron microscope (TEM) analysis were prepared by fluorescence-assisted cell sorting (FACS) in the same manner as for flow cytometry analysis. Cells labeled with anti CD8⁺ (FITC) antibody were separated from the splenocyte population using a BD FACSAria™ cell sorter.

Results: Targeting nanoparticles showed significant CTL binding (58.47%; note that only a specific CTL subpopulation is targeted) and minimal non-CTL attachment (9.33%), demonstrating selective cell labeling. CTL labeling efficiency was measured as the ratio of CTLs labeled by nanoparticles or tetramers divided by the total CTL population (CD8⁺ cells) (Table 1). Targeting nanoparticles demonstrated 3.9-fold higher labeling of CTLs than non-targeting nanoparticles and 44-fold higher labeling efficiency for CTLs than for non-CTLs. Non-targeting nanoparticles showed only 15% of the CTLs labeled, which is normal as a result of non-specific particle attachment. The targeting nanoparticle bound to CTLs with the complementary TCR, while non-targeted CTLs did not bind the nanoparticles (1.34% labeled; FIG. 4B), further demonstrating the specificity of targeting nanoparticles.

TABLE 1
Percentage of CTLs labeled by nanoparticles, as evaluated by
flow cytometry.

| | Splenocytes without CTL | Splenocytes with CTL (from |
|---|---|---|
| | (from w.t. B6 mice) (%) | pmel-1 mice) (%) |
| Non-Targeting | 0.34 | 15.13 |
| Nanoparticles | | |
| Targeting | 1.34 | 58.47 |
| Nanoparticles | | |
| Tetramer | 0 | 49.46 |

### Example 8

### NP-PEG-MHC-AF647 Nanoparticles as an MRI Probe

Isolated splenocytes were incubated with either CTL-targeting (anti-CD8 antibody coated) or non-CTL-targeting magnetic nanoparticles (specific to alternative cell markers; Miltenyi, Auburn, Calif.). Each population was passed through an autoMACS™ magnetic column to remove labeled cells and separate untouched CTLs and non-CTLs. These cells were incubated with peptide-MHC-conjugated nanoparticles for 3 hrs, washed 3× with PBS, and equilibrated to 1.5 million cells per sample. Cells were suspended in an agarose cast and visualized with a 4.7-T Varian MR spectrometer (Varian, Inc., Palo Alto, Calif.) and a Bruker magnet (Bruker Medical Systems, Germany) equipped with a 5-cm volume coil. A spin-echo multisection pulse sequence was selected to acquire MR phantom images. Repetition time (TR) of 3000 msec and variable echo times (TE) of 15-90 msec were used. The spatial resolution parameters were as follows: an acquisition matrix of 256×128, field of view of 4×4 cm, section thickness of 1 mm, and 2 averages. Regions of interest (ROIs) of 5.0 mm in diameter were placed in the center of each sample image to obtain signal intensity measurements using NIH ImageJ. T2 values were obtained using VnmrJ “t2” fit program to generate a T2 map of the acquired images. Cells incubated with peptide-MHC labeled nanoparticles were imaged with a Philips CM100 TEM at 100 kV with a Gatan 689 digital slow scan camera.

The MR phantom image in FIG. 4C shows the CTLs significantly darker (negative contrast enhancement) than the non-CTL cells. The contrast enhancement was quantified by the corresponding T2 relaxation times, which were 24±3 ms and 71±2 ms for CTL and non-CTL samples, respectively. Specific cell labeling, here, was markedly more efficient (0.5-3 hr) than alternative non-specific loading schemes that require relatively lengthy incubation times (up to 48 hours).

### Example 9

### Avidity Study Using NP-PEG-MHC-AF647 Nanoparticles

To test the avidity of NP-PEG-MHC-AF647 (Example 4) for CTLs, cell-binding of the targeting nanoparticle was compared with that of MHC-tetramer-AF647 (Example 5). Flow cytometry (see Example 7) showed that the nanoparticle binding to the CTLs was higher than tetrameric labeling (59.4% vs. 46.3%; FIG. 4D), probably attributable to higher valency of the nanoparticles. Although four peptide-MHC complexes are presented on each tetramer, steric hindrance limits the number of bound complexes to two or three at a time. Nanoparticle labels are expected to offer greater binding avidity due to increased peptide-MHC presentation. The multiple, flexible PEG chains of the nanoparticle coat, on which the targeting molecule is displayed, can present multiple peptide-MHCs to the target cell.

### Example 10

### Specificity Study Using NP-PEG-MHC-AF647 Nanoparticles

Prolonged cell exposure to nanoparticles may potentially increase non-specific particle attachment to cells. In particular, while the PEG coating on nanoparticles limits unwanted interactions, a small fraction of cells eliciting nonspecific nanoparticle association is not unexpected. To maintain minimal particle-cell interaction outside of the MHC/peptide presentation, neutravidin was exploited for its low isoelectric point and the lack of an expressed RYD sequence (present in streptavidin). To show minimal non-specific interactions, splenocytes were incubated with nanoparticles for 1 or 3 hrs. The results in FIGS. 4E and 4F show that targeting nanoparticles showed 4.65 times higher avidity for CTLs than non-targeting nanoparticles after 1 hr, and 5.54 times after 3 hrs. Significantly, non-specific attachment of non-targeting nanoparticles remained under 14% after 3 hours.

Further, targeted nanoparticle labeling was high (74%) after incubation for 3 hrs compared to alternative loading schemes that require incubation times of over 24 hrs, indicating efficient cell tagging.

### Example 11

### Targeting Cellular Labeling Using NP-PEG-MHC-AF647 Nanoparticles

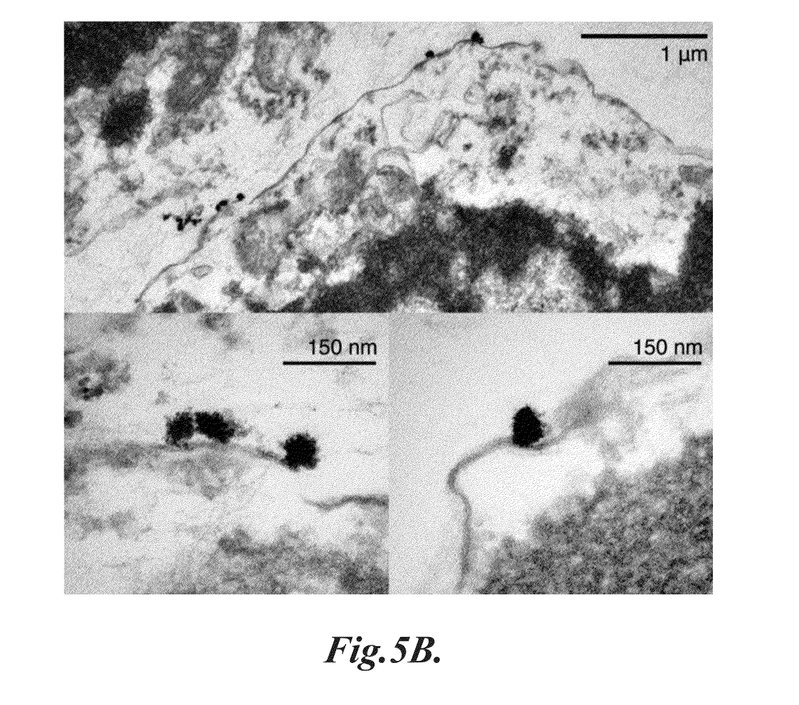

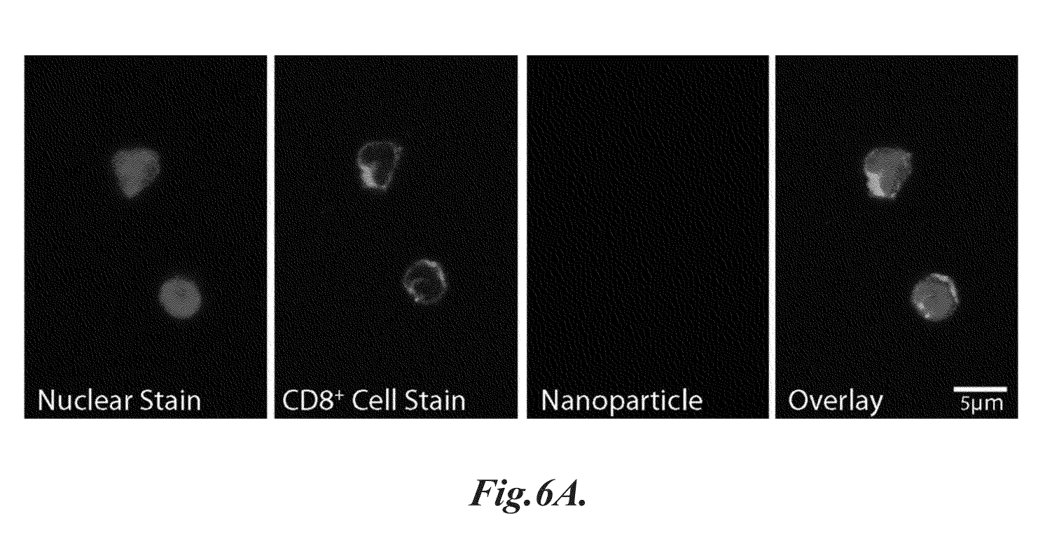

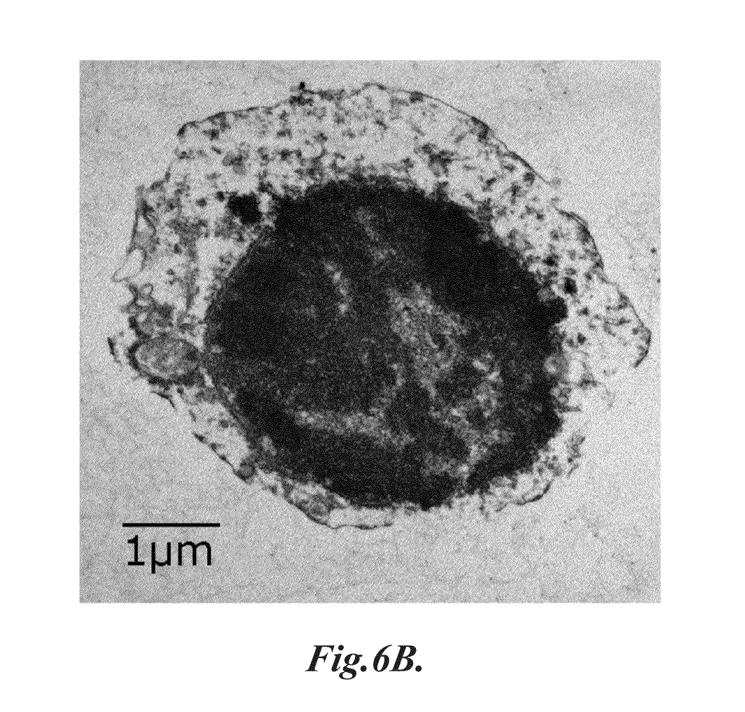

Targeted cellular labeling with the nanoparticles and their cellular localization was visualized by fluorescence microscopy. Splenocytes containing CTLs were incubated with targeting nanoparticles (NP-PEG-MHC-AF647) for 1 hr (see Example 7) and 2×10⁵ cells were plated on cover slips and fixed with a 4% paraformaldehyde solution. After fixation, cells were stained with 4′,6-diamidino-2-phenyindole (DAPI) per the manufacturer's instructions and imaged. Confocal images were acquired on a DeltaVision® SA3.1 wide-field deconvolution microscope (Applied Precision, Inc., Issaquah, Wash.) with DAPI and Cy5 filters (emission: 655 nm). SoftWoRx (Applied Precision, Inc.) was used for image processing, including normalization of fluorescence intensity. Fluorescence images of cells co-stained with the CD8⁺ antibody (green), DAPI nuclear stain (blue), and nanoparticles labeled with the AF647 (red) demonstrated specific attachment of the targeting nanoparticles to CTL cells (FIG. 5A), while non-targeting nanoparticles (NP-PEG-AF647) showed limited AF647 fluorescence signal from either CD8⁺ or CD8⁻ cells (FIG. 6A).

The localization of nanoparticles within the cells was examined by transmission electron microscopy (TEM). Splenocytes containing CTLs were incubated with targeting nanoparticles; the CTL subpopulation was then isolated by fluorescence-activated cell sorting (FACS) and imaged by TEM. Nanoparticles accumulated at the outer leaflet of the cell membrane (FIG. 5B), agreeing with the fluorescence imaging where signal intensity was greatest at the cellular edges indicating surface localization of nanoparticles (FIG. 5A). The specific attachment of individual nanoparticles at the cellular membrane illustrates the selective binding of the nanoparticles via TCR affinity. Binding of non-targeting nanoparticles to CTLs was not readily observed by TEM (FIG. 6B), further verifying the flow cytometry studies.

### Example 12

### Analysis of Functional CTL Activity Following Labeling with NP-PEG-MHC-AF647 Nanoparticles

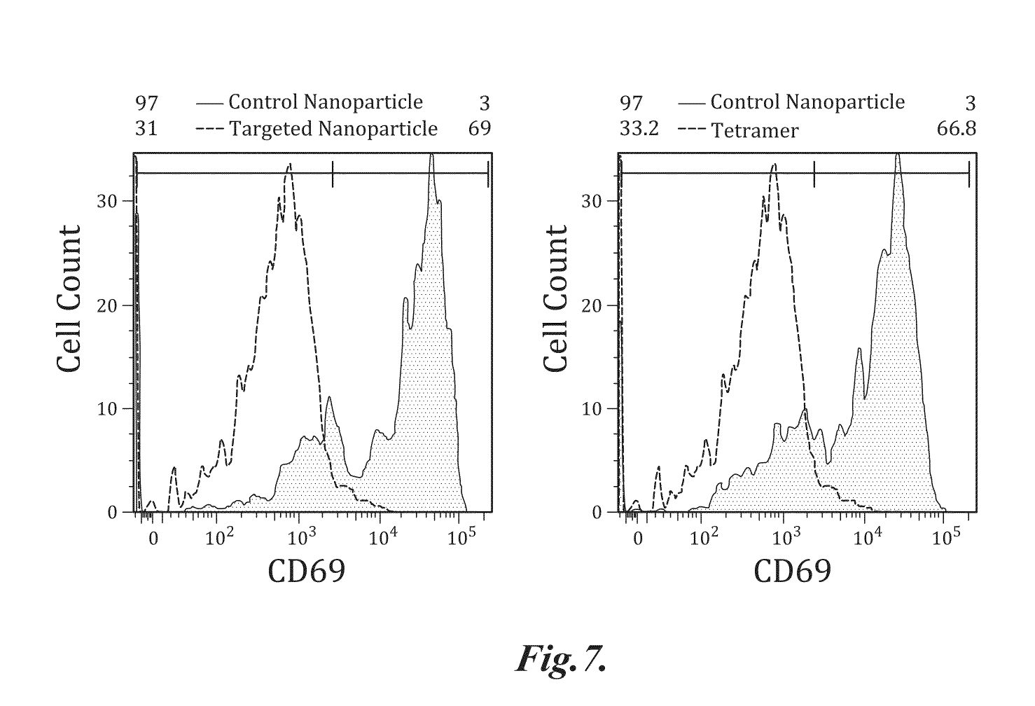

Functional CTL activity after nanoparticle labeling with NP-PEG-MHC-AF647 nanoparticles was demonstrated by the upregulation of the activation induction molecule (CD69; FIG. 7). CD69 expression was characterized by flow cytometry 18 hrs post incubation on CTLs exposed to targeting nanoparticles, non-targeting nanoparticles, or peptide-MHC tetramer. Study showed cells exposed to either targeting nanoparticles or MHC-tetramer-AF647 tetramers elicited similar, normal CD69 expression (69 and 66.8%, respectively), while unstimulated control nanoparticles demonstrated no CD69 increase (FIG. 7).

The embodiments of the invention in which an exclusive property or privilege is claimed are defined as follows:

1. A method of detecting the presence of cells having a T cell receptor in a sample, comprising:
(a) contacting the sample with a nanoparticle comprising:
(i) a core comprising a magnetic material and having a surface; and
(ii) an antigenic peptide-major histocompatibility complex (MHC) monomer operatively linked to the surface, wherein the antigenic peptide-MHC monomer is recognized by the T cell receptor; and
(b) measuring the level of nanoparticle binding to cells in the sample using magnetic resonance imaging.

2. The method of claim 1, wherein the cells are tumor cell-specific cytotoxic T cells.

3. The method of claim 1, wherein the cells are in vitro, ex vivo, or wherein the sample is a tissue.

4. The method of claim 1, wherein the magnetic material is selected from the group consisting of ferrous oxide, ferric oxide, silicon oxide, polycrystalline silicon oxide, silicon nitride, aluminum oxide, germanium oxide, zinc selenide, tin dioxide, titanium, titanium dioxide, indium tin oxide, gadolinium oxide and stainless steel.

5. The method of claim 1, wherein the magnetic material is a doped nanoparticle.

6. The method of claim 5, wherein the doped nanoparticle is selected from the group consisting of nickel titanium, MnFeO₄, CoFe₂O₄, and NiFe₂O₄.

7. The method of claim 1, wherein the antigenic peptide is selected from the group consisting of pmel-1, HA-1, MART-1, gp100, NY-ESO-1, WT-1, GAD65, CMV pp65, EBNA, LMP2, HIV-gag, BCR-ABL, Mart2, Mum-1, Mum-2, Mum-3, Bage-1, Gage 3, Gage 4, Gage 5, Gage 6, Gage 7, GnTV, Herv-K-mel, Lage-1, Mage-A1, Mage-A 2, Mage-A 3, Mage-A 4, Mage-A 6, Mage-A 10, Mage-A 12, Mage-C2, NA-88, SP17, SSX-2, TRP2-Int2, TRP-1, TRP-2, MACE-1, p15(58), CEA, RAGE, SCP-1, Hom/Mel-40, PRAME, HER-2/neu, E2A-PRL, H4-RET, IGH-IGK, MYL-RAR, human papillomavirus (HPV) antigens E6 and E7, TSP-180, p185erbB2, p180erbB-3, c-met, nm-23H1, PSA, TAG-72-4, CA 19-9, CA 72-4, CAM 17.1, NuMa, p16, TAGE, PSMA, PSCA, CT7, telomerase, 43-9F, 5T4, 791Tgp72, 13HCG, BCA225, BTAA, CA 125, CA 15-3, CA 27.29, CA 195, CA 242, CA-50, CAM43, CD68\KP1, CO-029, FGF-5, G250, Ga733 (EpCAM), HTgp-175, M344, MA-50, MG7-Ag, MOV18, NB\70K, NY-CO-1, RCAS1, SDCCAG16, TAAL6, TAG72, TLP, DKK1, EZH2, ALDH1A1, and TPS.

8. The method of claim 1, wherein the nanoparticle further comprises a polymer that forms a coating on the surface, and the antigenic peptide-MHC monomer is operatively linked to the polymer.

9. The method of claim 8, wherein the nanoparticle has a hydrodynamic size of about 5-300 nm.

10. The method of claim 8, wherein the polymer that forms a coating on the surface is covalently bound to the surface.

11. The method of claim 8, wherein the polymer that forms a coating on the surface is physically adsorbed to the surface.

12. The method of claim 8, wherein the polymer is selected from the group consisting of poly(ethylene glycol) (PEG), chitosan, and chitosan-PEG.

13. The method of claim 8, wherein the polymer is covalently bound to an avidin protein.

14. The method of claim 13, wherein the antigenic peptide-MHC monomer further comprises biotin and is bound to the avidin protein through a biotin/avidin interaction.

15. The method of claim 1, further comprising a signal-generating label.

16. The method of claim 15, wherein the signal-generating label is a fluorophore, a chromophore, or a radiolabel.

17. A method of detecting the presence of cells having a T cell receptor in a subject, comprising:
(a) administering to the subject a nanoparticle comprising:
(i) a core comprising a magnetic material and having a surface; and
(ii) an antigenic peptide-major histocompatibility complex (MHC) monomer operatively linked to the surface, wherein the antigenic peptide-MHC monomer is recognized by the T cell receptor; and
(b) measuring the level of nanoparticle binding to cells in the subject using magnetic resonance imaging.

18. The method of claim 17, wherein administration is by intravenous injection or intratumoral injection.

19. A method of detecting the presence of cells having a T cell receptor in a sample, comprising:
(a) contacting the sample with a nanoparticle comprising:
(i) a core comprising a magnetic material and having a surface;
(ii) an antigenic peptide-major histocompatibility complex (MHC) monomer operatively linked to the surface, wherein the antigenic peptide-MHC monomer is recognized by the T cell receptor; and
(iii) a fluorophore;
(b) isolating those cells from the sample that bound to the nanoparticle; and
(c) measuring the level of nanoparticle binding to cells in the sample using fluorescence detection.
